(19) United States
(12) Patent Application Publication
(10) Pub. No.: US 2011/0276095 A1
(43) Pub. Date: Nov. 10, 2011

(54) Double Threaded Orthopedic Screw

(76) Inventors: Yossef Bar, Tirat HaCarmel (IL); Eli Zehavi, Haifa (IL); Brian Hewko, Bay Village, OH (US); Moshe Shoham, Hamovil (IL)

(21) Appl. No.: 13/133,657

(22) PCT Filed: Dec. 9, 2009
(86) PCT No.: PCT/IL09/01169

§ 371 Date: Jul. 27, 2011

Related U.S. Application Data
(60) Provisional application No. 61/193,586, filed on Dec. 9, 2008.

(51) Int. Cl. A61B 17/88; A61B 17/86

(52) U.S. Cl. 606/279; 606/315

(57) ABSTRACT

An orthopedic screw having a thread with two parts, a distal and a proximal part, each having a different thread configuration. The distal section has a thread with outer dimension and pitch suitable for entry into cancellous bone, while the proximal section has a composite thread comprising (i) a first thread of the same or slightly larger outer diameter as the cancellous thread in the distal section, having the same pitch thereof, and lying on the same helix, and (ii) another thread having a smaller outer diameter but the same pitch as the first thread, but disposed on a helix displaced from that of the first thread, such that it lies between the crests of the first thread. This screw enables optimum fixation strength in a bone or bones having a harder cortical outer section and a softer cancellous inner section. The screw may have an unthreaded central section.

### FIELD OF THE INVENTION

The present invention relates to the field of orthopedic screws for use in joining bones, especially for use in situations where two types of bone need to be penetrated and joined.

### BACKGROUND OF THE INVENTION

A common treatment for spinal stabilization is the fixation of two or more vertebrae performed by insertion of a pair of screws into each of the vertebrae to be fused and connecting the screws heads on either side of the spine by two rigid rods. Cleaning the disc space and inserting bone graft into the cleaned disc space causes bone to grow between the vertebrae until, several months later, the fusion is completed.

The screws are usually inserted into the pedicles, two for each vertebra such that a minimum of four screws are required for each level of fusion. Reference is now made in FIG. 1 which is a cross-sectional axial view of a vertebra 10, showing a pair of pedicle screws 11 passing from the pedicles 12 into the vertebral body 13, and clamping the fusion rods 14 to the vertebrae. As is observed the screws must pass from the hard cortical bone of the pedicle into the comparatively soft cancellous bone 15 of the vertebral body. Spinal fusion by means of pedicle screw insertion is currently the most common procedure adopted for spinal stabilization, with hundreds of thousands of cases performed each year all over the world. Though most of these procedures are performed using open back surgery, recent improvements in surgical techniques have enabled the procedure to be performed less invasively. In particular, computer guided robotic techniques have been developed to ensure the accuracy required for screw insertion.

A different fixation technique, using only two obliquely inserted screws, one on either side of the spine, was first described in the article entitled “Direct Pediculo-Body Fixation in Cases of Spondylolisthesis with Advanced Intervertebral Disc Degeneration”, European Spine Journal, Vol. 5, pp. 281-285; 1996, by D. Grob et al. The surgical approach suggested in this article is for oblique trans-pedicular interbody fixation at the L4-L5 and L5-S1 levels. In this technique, a pair of screws is inserted bilaterally through the pedicles of the inferior vertebra and passed diagonally across the disc space towards the anterior cortical rim of the superior vertebral body. Variations of such an oblique process have been described again in US Patent Application Publication No. US 2009/0163957 for “Oblique Lumber Interbody Fusion” assigned to the Cleveland Clinic Foundation, and in co-pending PCT Patent Application No. PCT/IL2009/001130 entitled “Robot Guided Oblique Spinal Stabilisation”, to Mazor Surgical Technologies Ltd., where there is described a system and method for performing a minimally invasive oblique entry spinal fusion procedure by use of a robotic surgical system, yet with minimal danger to nerve structures in the vicinity of the entry trajectories. Reference is now made in FIG. 2 which is a schematic lateral view of two adjacent vertebrae 21, 22, illustrating the position and path of entry of an obliquely inserted interbody fixation screw 23. Also in this procedure, the screw 23 must pass from the hard cortical bone of the pedicle region 24, through the hard cortical bone of the facing endplates 25, 26 of the inferior and superior vertebrae, and into the soft cancellous bone 28 of the superior vertebra body.

Both of these spinal fusion methods involve screw trajectories which, from the entry point at the pedicle through to the vertebral body, pass through two different and separated types of bone structures. It is known that in order to provide good support and high pull-out resistance in cancellous bone, a coarser pitch screw must be used, in comparison with a screw which has to penetrate the considerably harder cortical bone. The prior art screws used in these procedures are thus essentially a compromise between optimum fixation in cancellous bone, and the same for cortical bone entry and fixation.

The disclosures of each of the publications mentioned in this section and in other sections of the specification, are hereby incorporated by reference, each in its entirety.

### SUMMARY OF THE INVENTION

The present disclosure describes new exemplary orthopedic fixation screws for use in different types of bone. Since orthopedic fixation screws may penetrate and hold bone structures having different properties, the screws should be constructed accordingly to take into account these different properties. Thus, the conventional posterior entry pedicle screw has to go through different bone layers while entering from the pedicle into the vertebral body. Likewise, the oblique screw has to go through different bone layers while entering from the inferior vertebrae pedicle, through the disc-space, and then into the superior vertebra body. In either case, the distal end of the screw must first go through the cortical bone layers of the pedicle walls—but that same distal end must still be designed to have the optimal grip in the cancellous bone of the vertebra body.

One of the main features of the exemplary screws described in this application is the need to sustain pull-out forces. Since cancellous bone is softer than cortical bone, a coarser thread is optimally used for the cancellous bone than for the cortical bone. The screw should thus contain two different threads—one that is optimized for use in cancellous bone at the distal end and one that is optimized for use in cortical bone at the proximal end.

Maximizing pull-out force requires as large a screw diameter as possible especially at the distal tip where the screw is inserted into the less rigid cancellous bone. However, for the spinal fusion procedures, maximum screw diameter is limited by the 5-8 mm pedicle wall diameter through which the screw has to pass before reaching cancellous bone of the vertebral body.

The exemplary screws described in this application should have increased pull-out force when inserted, yet without increasing the insertion torque and hence the forces acting on the vertebrae during the screw insertion. Also the screws should be constructed so as to have maximum strength against compression and bending failure.

One exemplary implementation involves a screw for orthopedic use, the screw comprising:

(i) a distal section comprising a distal screw thread having an outer diameter and a pitch, and
(ii) a proximal section comprising a double screw thread, the double screw thread having a first screw thread having the same pitch and formed on the same helix as that of the distal screw thread, and a second screw thread having the same pitch but a smaller outer diameter than that of the distal screw thread, and formed on a different helix such that its threads are disposed intermediate the threads of the first screw thread.

In such a screw, the first screw thread of the proximal section may have essentially the same outer diameter as that of the distal screw thread, or it may have a larger outer diameter than that of the distal screw thread. The helix of the second screw thread may advantageously be offset from the helix of the first screw thread by half of the pitch of the distal screw thread, such that the crests of the second screw thread fall essentially midway between the crests of the first screw thread.

Additional implementations can include a screw as described above, and further comprising an unthreaded section disposed between the distal section and the proximal section. This unthreaded section may have essentially the same diameter as the core diameter of the distal screw thread such that the unthreaded section provides support to the screw within a bore drilled in the bone for the screw.

In any of the above-described screws, the distal screw thread may have parameters selected to provide efficient fixation in cancellous bone, and the second screw thread of the proximal section may have parameters selected to provide efficient fixation in cortical bone.

Still another exemplary implementation may involve a method of affixing a screw in a bone structure having a cortical outer region, and a cancellous inner region, comprising:

(i) providing a screw having a distal section comprising a distal screw thread having an outer diameter and a pitch, and a proximal section comprising a double screw thread, the double screw thread having a first screw thread having the same pitch and formed on the same helix as that of the distal screw thread, and a second screw thread having the same pitch but a smaller outer diameter than that of the distal screw thread, and formed on a different helix such that its threads are disposed intermediate the threads of the first screw thread,
(ii) preparing a hole in the bone in which the screw is to be affixed, and
(iii) inserting the screw into the hole such that the distal section of the screw is affixed at least part in the cancellous bone, and the proximal section of the screw is affixed at least part in the cortical bone.

In this method, the first screw thread of the proximal section should have at least the same outer diameter as that of the distal screw thread. Additionally, the helix of the second screw thread may be offset from the helix of the first screw thread by half of the pitch of the distal screw thread, such that the crests of the second screw thread fall essentially midway between the crests of the first screw thread. In any of the above methods, the screw may further comprise an unthreaded section disposed between the distal section and the proximal section.

A further exemplary method described in this disclosure is that of affixing an inferior vertebra having a pedicular region with a cortical outer region, to the body of a superior vertebra, the vertebral body having a cancellous inner region, the method comprising:

(i) providing a screw having a distal section comprising a distal screw thread having an outer diameter and a pitch, a central unthreaded section, and a proximal section comprising a double screw thread, the double screw thread having a first screw thread having the same pitch and formed on the same helix as that of the distal screw thread, and a second screw thread having the same pitch but a smaller outer diameter than that of the distal screw thread, and formed on a different helix such that its threads are disposed intermediate the threads of the first screw thread,
(ii) preparing a passage for the screw from the pedicular region in the inferior vertebra to the body region of the superior vertebra, and
(iii) inserting the screw into the passage such that the distal section of the screw is affixed at least part in the cancellous bone, the proximal section of the screw is affixed at least part in the cortical bone, and the unthreaded section is located in the disc space between the vertebrae.

In this latter method, the first screw thread of the proximal section should have at least the same outer diameter as that of the distal screw thread. Additionally, the helix of the second screw thread may be offset from the helix of the first screw thread by half of the pitch of the distal screw thread, such that the crests of the second screw thread fall essentially midway between the crests of the first screw thread.

Although the screws described in this application have been described generally in relation to spinal fusion procedures, it is to be understood that this application is not meant to be limited to such cases but that the screws described and claimed could be used in any orthopedic procedure where it is necessary to join different bone structures or different parts of bone structures. In general, bones have a hard cortical outer layer, and a softer cancellous bone interior, such that almost any orthopedic screw may be able to benefit by using the thread construction described in this disclosure.

### BRIEF DESCRIPTION OF THE DRAWINGS

The presently claimed invention will be understood and appreciated more fully from the following detailed description, taken in conjunction with the drawings in which:

FIG. 1 showed a cross-sectional axial view of a vertebra with prior art spinal fixation screws inserted;

FIG. 2 showed schematic lateral view of two adjacent vertebrae illustrating the position and path of entry of a prior art obliquely inserted interbody fixation screw;

FIG. 3 illustrates schematically a first exemplary bone screw having a composite thread with two distinct parts, a distal part and a proximal part, each having a different thread configuration;

FIG. 4 illustrates schematically a second exemplary bone screw having an unthreaded section between the distal and proximal parts of the screw;

FIG. 5 is a cross sectional enlarged drawing of the tooth profile of the thread in the distal part of the screws of FIGS. 3 and 4; and

FIG. 6 is a cross sectional enlarged drawing of the tooth profile of the thread in the proximal part of the screws of FIGS. 3 and 4.

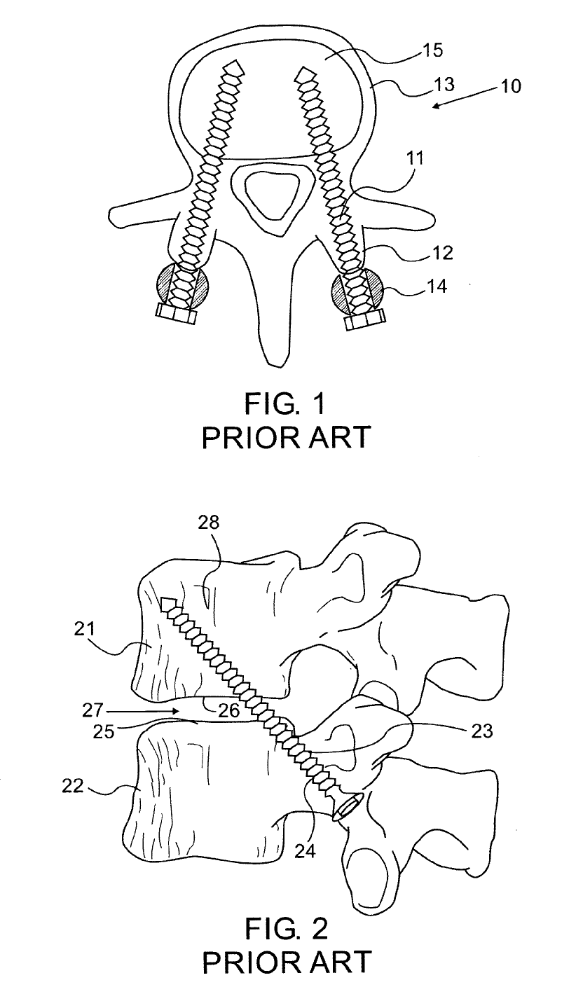

### DETAILED DESCRIPTION

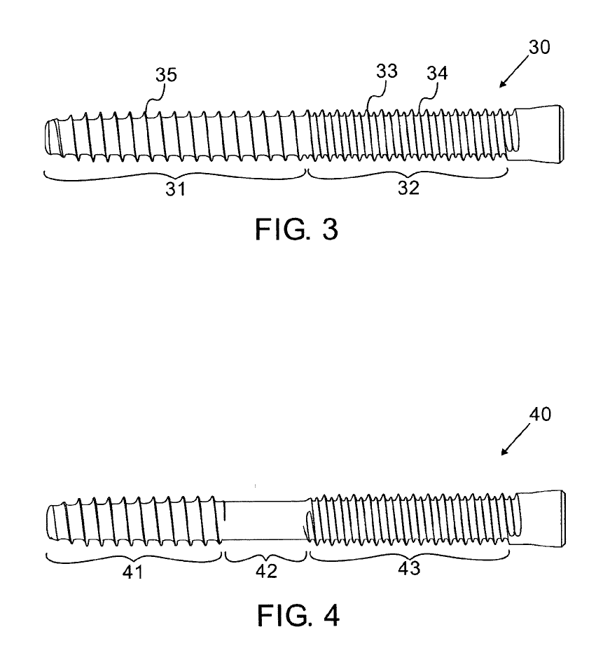

Reference is now made to FIG. 3, which illustrates schematically a bone screw 30 according to a first implementation of the screws described in this application. The thread of the screw has two distinct parts, a distal part 31 and a proximal part 32, each having a different thread configuration:

(i) a thread 35 at the distal end, optimally sized and pitched for entry and fixation in cancellous bone, and
(ii) a double thread configuration at the proximal end 32, for entry and fixation in cortical bone, with two threads having different outer diameters, a larger 33 and a smaller 34 outer diameter.

In this proximal section, the larger, outer diameter thread 33:

(a) has the same pitch as the pitch of the cancellous thread 35 of the distal section,
(b) has an outer diameter which is generally the same or slightly larger, but not less than that of the cancellous thread 35 of the distal section, and
(c) is formed on the same helix as that of the cancellous thread 35 of the distal section.

The smaller outer diameter thread 34 also has the same pitch as the pitch of the cancellous thread 35 of the distal section, but the thread outer (crest) diameter is smaller, and the threads are formed on a different helix, offset from the larger diameter thread helix generally by half the pitch, such that the smaller diameter thread crests fall generally midway between the larger diameter thread crests.

In use, as the screw is inserted, the distal end 31 with the cancellous self-tapping thread 35 will first go through the drilled hole in the pedicle cortical walls of the vertebra, cutting an internally tapped thread matched in pitch to the cancellous thread part of the screw. As the screw progresses and the distal end reaches the vertebra cancellous body 15, generating its internally threaded hole therein, the proximal end composite thread 32 now enters the pedicle, and its larger diameter threads 33 screw exactly into the previously tapped cancellous threads cut by the distal end thread. Since the threads have been precut in the pedicle by the distal end, only minor additional torque is required for insertion of the larger diameter proximal threads into the pedicle hole.

On the other hand, the smaller diameter thread 34 in the proximal part 32 of the screw does generate a new additional thread cut into the cortical bone of the pedicle, positioned between the internal crests of the already cut larger diameter thread and since the cortical bone is harder than the cancellous bone, even this reduced size thread does provide significant additional grip for the screw, thereby increasing the overall pull-out force of the screw within the combined bone structure. The generation of the low threads does add some additional torque during insertion, but because of the smaller dimensions of the smaller diameter thread, this additional torque does not add substantially to that required to generate the cancellous thread in the body of the superior vertebra. In any event, if the larger diameter thread 33 is the same diameter as that of the cancellous distal thread 35, the precut large diameter thread form in the pedicle does not add any significant torque during the screw insertion.

As previously mentioned, this larger diameter thread 33 in the proximal part of the screw should not have an outer diameter smaller than that of the cancellous thread of the distal part of the screw, since this would result in a looser fit when the proximal part of the screw is driven home into the pedicle. On the other hand, if it has a slightly larger outer diameter than that of the cancellous thread 35 of the distal part of the screw, although there will be a slightly increased insertion torque generated as the proximal part of the screw is inserted, the small additional thread cut into the cortical bone by the larger diameter screw thread 33, in addition to the smaller diameter thread cut therein, generally adds to the pull-out force of the screw within the vertebral bone structure. However, the outer diameter of this thread form 33 should not be so large as to compromise the cortical bone structure, such as by breaking out of it.

Any of the thread forms may be produced with a taper, in order to assist in the self tapping insertion action of the screw into the bone. In particular, according to one exemplary implementation, the distal part 31 of the screw may have a taper of the order of 2° or so to assist with the insertion of the cancellous thread 35. This taper may be only on the core diameter of the screw, as shown in FIG. 3, or on both the core and outer diameters.

Reference is now made to FIG. 4, which illustrates schematically a bone screw 40 for preferred use in an oblique entry vertebral stabilization procedure. The screw has three distinct parts—a distal part 41, a smooth central part 42, and a proximal part 43, the distal and proximal parts each having a different thread configuration. The distal and proximal thread configurations may be the same as those of the screw shown in FIG. 3. This screw is thus similar in structure, except for the presence of the central clear section 42 having no thread. This central area is positioned such that when the screw is fully inserted, it is located in the disc space 27 between the vertebral bodies, where no screw thread is required, and even if there were one, it would not contribute to the pull out force of the screw. Additionally, for those applications other than vertebral stabilization, where the screw is used in a single bone structure, since the central unthreaded section 42 has the same outer diameter as the core diameter of the screw, and since this is generally the diameter of the hole drilled in the bone to take the screw, this central section sits tightly in the bone bore, providing additional lateral support.

There is an additional advantage obtained by the presence of the blank central section of the screw of FIG. 4 compared with that of FIG. 3. As is known in the art, screw fatigue failure generally takes place near the center of the screw. The machining or grinding of the screw thread generates microscopic stress raisers in the surface of the screw thread, from which fatigue cracks can propagate. The elimination of the screw cutting procedure at the central section of the screw of FIG. 4 contributes to an increase of the fatigue breaking stress of the screw, as compared to a screw with a thread all the way along its length. Fatigue texts were performed on otherwise identical screws having the form of that of FIG. 3 and that of FIG. 4. While the continuous thread screws failed after approximately 20,000 stress cycles, the screw with the clear unmachined center withstood almost 100,000 cycles before failing. Furthermore, unlike most orthopedic screw fixations, where the screw is fully supported along its length within the bore in the bone, in an oblique entry intervertebral fusion process, the screw is not supported by bone in the disc space, or at least not until bone graft material has fully grown in the disc space. Consequently, the load on the screw is substantial across the disc space, and the screw should have maximum strength in this region. This is a further reason for having, for such use, as large a diameter and as clear a central section as possible.

Tests were also performed to ascertain the improvement in static pull-out force resulting from the use of the composite thread form, whether in screws of the type of FIG. 3 or of FIG. 4. The tests were performed according to the ASTM F543 Static Pull-out and Driving Torque Test Procedure, on orthopedic screws having continuous threads, like those of FIG. 3. The results were compared for the axial pull-out strength from a pair of blocks of Grade 15 polyurethane foam, used to simulate cancellous bone. Conventional orthopedic type 100 Series screws supplied by Mazor Surgical Technologies of Caesarea, Israel were compared with comparatively sized screws of the 700 series by the same manufacturer, the 700 series having the composite thread form as described in this application.

An increase in pull out force of approximately 11% was found for the composite thread screws of the 700 series, as per the present disclosure. Additionally, a decrease in the peak insertion torque of approximately 10% was found for the composite thread screw of the present disclosure. Both these results indicate the usefulness of the new composite thread form screws described in this application, wherein the pull-out force is increased, yet without a corresponding increase in the insertion torque, which would be disadvantageous for orthopedic use.

The major diameter of the screw, and the tooth shape can be used to adapt the screws for specific applications. For use in oblique entry vertebral stabilization, a 7 mm diameter by 70 mm long screw may advantageously be used, with a 2 mm pitch. In order to readily penetrate the cortical/space/cortical/cancellous progression of bone structures for this procedure, the outer diameters of the thread may be 6.75 mm in the distal region 35, and 7 mm in the proximal region 34, with a major diameter of 5.25 for the small diameter proximal section thread 33.

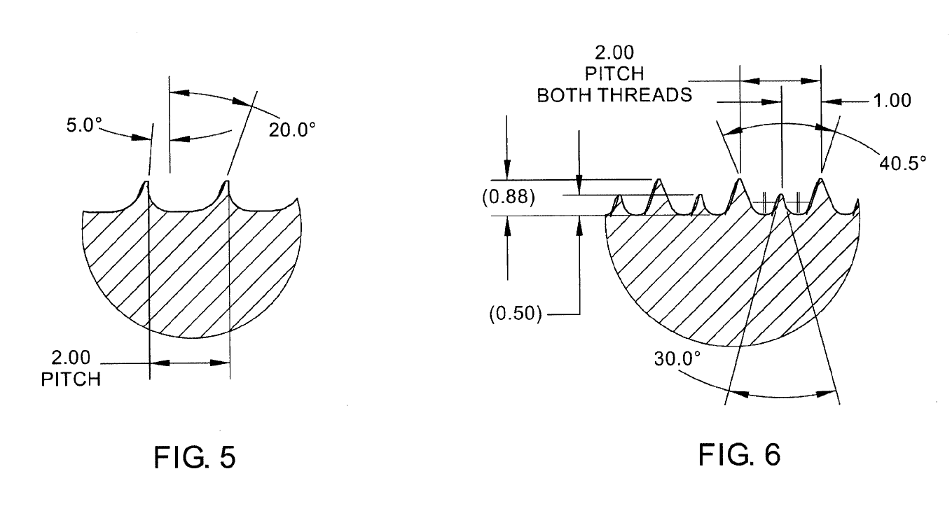

Reference is now made to FIGS. 5 and 6, which illustrate schematically cross sectional enlarged drawings of the tooth profiles of examples of possible threads in the distal (FIG. 5) and the proximal (FIG. 6) parts of the screws such as those shown in FIGS. 3 and 4. The distal tooth profile shown in FIG. 5 is shaped with a 5° undercut trailing edge flank near its crest, such that it provides good fixation in the soft cancellous bone into which it is intended to be driven.

Referring now to FIG. 6, it is seen that the larger outer diameter thread 33 of the proximal part is shown to have a 40° symmetrical included angle, such that for this example screw, this tooth takes an additional slight cut as it is inserted into the cortical bone along the path of the distal thread. The smaller outer diameter thread 34 has an included angle of 30°, but the tooth height is only about 55% of the height of the tooth of the larger outer diameter thread 33. Although the tooth parameters shown in FIGS. 5 and 6 have been found to provide good performance in cancellous and cortical bone respectively, it is to be understood that they are only examples of possible configurations, and that other tooth shapes and dimensions may be used in these or other orthopedic situations without detracting from the novelty of the presently claimed invention.

It is appreciated by persons skilled in the art that the present invention is not limited by what has been particularly shown and described hereinabove. Rather the scope of the present invention includes both combinations and subcombinations of various features described hereinabove as well as variations and modifications thereto which would occur to a person of skill in the art upon reading the above description and which are not in the prior art.

1. A screw for orthopedic use, said screw comprising:
a distal section comprising a distal screw thread having an outer diameter and a pitch; and
a proximal section comprising a double screw thread, said double screw thread having a first screw thread having the same pitch and formed on the same helix as that of said distal screw thread, and a second screw thread having the same pitch but a smaller outer diameter than that of said distal screw thread, and formed on a different helix such that its threads are disposed intermediate the threads of said first screw thread.

2. A screw for orthopedic use according to claim 1 and wherein said first screw thread of said proximal section has essentially the same outer diameter as that of said distal screw thread.

3. A screw for orthopedic use according to claim 1 and wherein said first screw thread of said proximal section has a larger outer diameter than that of said distal screw thread.

4. A screw for orthopedic use according to claim 1 and wherein said helix of said second screw thread is offset from said helix of said first screw thread by half of the pitch of said distal screw thread, such that the crests of said second screw thread fall essentially midway between the crests of said first screw thread.

5. A screw for orthopedic use according to claim 1, further comprising an unthreaded section disposed between said distal section and said proximal section.

6. A screw for orthopedic use according to claim 5 wherein said unthreaded section has essentially the same diameter as the core diameter of said distal screw thread such that said unthreaded section provides support to the screw within a bore drilled in the bone for said screw.

7. A screw for orthopedic use according to claim 1, wherein said distal screw thread has parameters selected to provide efficient fixation in cancellous bone.

8. A screw for orthopedic use according to claim 1, wherein said second screw thread of said proximal section has parameters selected to provide efficient fixation in cortical bone.

9. A method of affixing a screw in a bone structure having a cortical outer region, and a cancellous inner region, comprising:
providing a screw having a distal section comprising a distal screw thread having an outer diameter and a pitch, and a proximal section comprising a double screw thread, said double screw thread having a first screw thread having the same pitch and formed on the same helix as that of said distal screw thread, and a second screw thread having the same pitch but a smaller outer diameter than that of said distal screw thread, and formed on a different helix such that its threads are disposed intermediate the threads of said first screw thread;
preparing a hole in said bone in which said screw is to be affixed, and
inserting said screw into said hole such that said distal section of said screw is affixed at least part in said cancellous bone, and said proximal section of said screw is affixed at least part in said cortical bone.

10. A method according to claim 9 and wherein said first screw thread of said proximal section has at least the same outer diameter as that of said distal screw thread.

11. A method according to claim 9 and wherein said helix of said second screw thread is offset from said helix of said first screw thread by half of the pitch of said distal screw thread; such that the crests of said second screw thread fall essentially midway between the crests of said first screw thread.

12. A method according to claim 9, wherein said screw further comprises an unthreaded section disposed between said distal section and said proximal section.

13. A method of affixing an inferior vertebra having a pedicular region with a cortical outer region, to the body of a superior vertebra, said vertebral body having a cancellous inner region, comprising:
providing a screw having a distal section comprising a distal screw thread having an outer diameter and a pitch, a central unthreaded section, and a proximal section comprising a double screw thread, said double screw thread having a first screw thread having the same pitch and formed on the same helix as that of said distal screw thread, and a second screw thread having the same pitch but a smaller outer diameter than that of said distal screw thread, and formed on a different helix such that its threads are disposed intermediate the threads of said first screw thread;
preparing a passage for said screw from said pedicular region in said inferior vertebra to said body region of said superior vertebra, and
inserting said screw into said passage such that said distal section of said screw is affixed at least part in said cancellous bone, said proximal section of said screw is affixed at least part in said cortical bone, and said unthreaded section is located in the disc space between said vertebrae.

14. A method according to claim 13 and wherein said first screw thread of said proximal section has at least the same outer diameter as that of said distal screw thread.

15. A method according to claim 13 and wherein said helix of said second screw thread is offset from said helix of said first screw thread by half of the pitch of said distal screw thread, such that the crests of said second screw thread fall essentially midway between the crests of said first screw thread.
